(19) United States
(12) Patent Application Publication
(10) Pub. No.: US 2008/0108683 A1
(43) Pub. Date: May 8, 2008

(54) 4-PHENYLTHIAZOLE AND 4-PHENYLIMIDIZOLE DERIVATIVES AND THEIR USE AS MEDICAMENTS FOR THE TREATMENT OF NEURODEGENERATIVE DISEASES, PAIN AND EPILEPSY

(75) Inventors: Pierre-Etienne CHABRIER de LASSAUNIERE, Paris (FR); Jeremiah Harnett, Gif-Sur-Yvette (FR); Dennis Bigg, Gif-Sur-Yvette (FR); Anne-Marie Liberatore, Auffargis (FR); Christophe Thurieau, Paris (FR); Zheng Xin Dong, Holliston, MA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques, Pairs Cedex (FR)

(21) Appl. No.: 11/928,884

(22) Filed: Oct. 30, 2007

Related U.S. Application Data
(62) Division of application No. 10/575,249, filed on Apr. 10, 2006.

(51) Int. Cl. A61K 31/4164; A61P 25/00; C07D 233/58

(52) U.S. Cl. 514/400; 548/338.1

(57) ABSTRACT

The invention relates to novel derivatives of 4-phenylthiazoles and 4-phenylimidizoles and the use thereof as medicaments. The invention especially relates to the following compounds: butyl-2-[4-(4-aminophenyl)-1H-imidizol-2-yl]ethyl-carbamate, and 4-[2-(1-aminocyclopentyl)-1,3-thiazol-4-yl]-2,6-di-tert-butylphenol, and the salts of the same.

The present invention relates to new 4-phenylthiazole and 4-phenylimidazole derivatives and their use as medicaments. Said medicaments are intended to inhibit monoamine oxydases (MAO) and/or lipidic peroxidation and/or to act as modulators of the sodium channels.

The compounds of the invention often present 2 or 3 of the activities mentioned above, which confer advantageous pharmacological properties on them.

In fact, taking into account the potential role of the MAO's and ROS's (“reactive oxygen species”, at the origin of lipidic peroxidation) in physiopathology, the new derivatives of the present invention can produce beneficial or favourable effects in the treatment of pathologies where these enzymes and/or these radicular species are involved. In particular:
disorders of the central or peripheral nervous system such as for example neurological diseases where Parkinson's disease, cerebral or spinal cord traumatisms, cerebral infarction, sub arachnoid hemorrhage, epilepsy, age ing, senile dementia, Alzheimer's disease, Huntington's chorea, amyotrophic lateral sclerosis, peripheral neuropathies, pain can in particular be mentioned;
schizophrenia, depressions, psychoses;
memory and mood disorders;
pathologies such as for example migraine;
behavioural disorders, bulimia and anorexia;
autoimmune and viral diseases such as for example lupus, AIDS, parasitic and viral infections, diabetes and its complications, multiple sclerosis.
addiction to toxic substances;
proliferative and inflammatory pathologies;
and more generally all the pathologies characterized by an excessive production of ROS's and/or participation of MAO's.

In all of these pathologies, experimental evidence exists which demonstrates the involvement of ROS's (Free Radic. Biol. Med. (1996) 20, 675-705; Antioxid. Health. Dis. (1997) 4 (Handbook of Synthetic Antioxidants), 1-52) as well as the involvement of MAO's (Goodman & Gilman's: The pharmacologial basis of therapeutics, 9th ed., 1995, 431-519).

The benefit of a combination of the inhibitory activities of MAO and inhibition of lipidic peroxidation is for example well illustrated in Parkinson's disease. This pathology is characterized by a loss of dopaminergic neurons of the nigrostriatal route the cause of which would in part be linked to an oxidizing stress due to ROS's. The exogenic dopamine from L Dopa is used in therapeutics in order to maintain sufficient levels of dopamine, MAO inhibitors are also used with L Dopa to avoid its metabolic degradation but do not act on the ROS's. Compounds which act both on MAO's and ROS's will therefore have a certain advantage.

Moreover, the character of the modulator of the sodium channels is very useful for therapeutic indications such as:
the treatment or prevention of pain, and in particular:
   postoperative pain,
   migraine,
   neuropathic pain such as trigeminal neuralgia, post-herpetic pain, diabetic neuropathies, glossopharyngeal neuralgias, secondary radiculopathies and neuropathies associated with metastatic infiltrations, adiposis dolorosa and pain associated with burns,
   central pain as a result of vascular cerebral accidents, thalamic lesions and multiple sclerosis, and
   chronic inflammatory pain or pain linked to a cancer;
the treatment of epilepsy;
the treatment of disorders linked to neurodegeneration, and in particular.
   vascular cerebral accidents,
   cerebral traumatism, and
   neurodegenerative diseases such as Alzheimer's disease, Parkinson's disease and amyotrophic lateral sclerosis;
the treatment of bipolar disorders and irritable bowel syndrome.

The concrete advantages of the presence in a compound of at least one of these activities is therefore clearly apparent from the above.

The European Patent Application EP 432 740 describes derivatives of hydroxyphenylthiazoles, which can be used in the treatment of inflammatory diseases, in particular rheumatic diseases. These derivatives of hydroxyphenylthiazoles show properties of trapping free radicals and inhibitors of the metabolism of arachidonic acid (they inhibit lipoxygenase and cyclooxygenase).

Other derivatives of hydroxyphenylthiazoles or hydroxyphenyloxazoles are described in the PCT Patent Application WO 99/09829. These have analgesic properties.

A certain number of derivatives of 4-phenylimidazole have moreover been described by the Applicant in the PCT Patent Application WO 99/64401 as agonists or antagonists of somatostatin. However, said derivatives of imidazoles have therapeutic properties in fields different from those indicated above (suppression of the growth hormone and the treatment of acromegalia, treatment of the recurrence of stenosis, inhibition of the secretion of gastric acid and prevention of gastro-intestinal bleeding in particular).

Moreover, the compounds of general formula (A1) 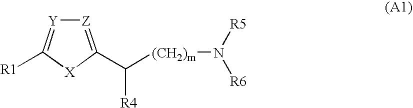
in which
R1 represents one of the aryl, heteroaryl, aralkyl or cycloalkyl radicals optionally substituted by one to three substituents chosen independently from a halogen atom, the CF₃, CN, OH, alkyl or alkoxy radical, SO₂R9 with R9 representing NH₂ or NHCH₃;
X represents NR2, R2 representing H or alkyl;
Y represents N or CR3;
Z represents CR3 or N;
on the condition however that Y and Z are not both CR3 or N at the same time;
R3 represents H, alkyl, halogen, hydroxyalkyl or phenyl optionally substituted by 1 to 3 substituents chosen from H, CF₃, CN, SO₂NH₂, OH, alkyl or alkoxy;
m represents 0, 1 or 2;
R4 represents H or alkyl;
when Z represents CR3, then R3 and R4 can also represent together —(CH₂)ₙ₁— with n1 an integer from 2 to 4 or R2 and R4 can also represent together —(CH₂)ₙ₂— with n2 an integer from 2 to 4;
R5 and R6 represent independently H, alkyl, alkoxy, aryl or aralkyl;
NR5R6 can also represent together (in particular):
the optionally substituted 2-(1,2,3,4-tetrahydroquinolyl) radical,
a radical 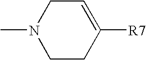
   in which R7 represents one of the phenyl, benzyl or phenethyl radicals in which the phenyl ring can be substituted;
a radical 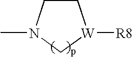
   in which p is an integer from 1 to 3,
   W is N and R8 represents H, CF₃, one of the phenyl, pyridyl or pyrimidinyl radicals optionally substituted once to twice by radicals chosen from halogen, OH, alkyl or alkoxy, or
   W is CH and R8 represents phenyl optionally substituted or aralkyl optionally substituted on the aryl group;
   have been described in the PCT Patent Application WO 96/16040 as partial agonists or antagonists of the dopamine sub-receptors of the brain or as prodrug forms of such partial agonists or antagonists. Therefore these compounds would have useful properties in the diagnosis and treatment of affective disorders such as schizophrenia and depression as well as certain disorders of movement such as Parkinson's disease.

It has also been described in the PCT Patent Application WO 98/27108 that certain amides of general formula (A2) 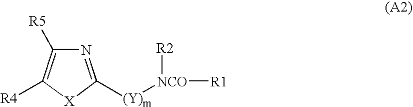
in which:
R1 represents in particular an alkyl, optionally substituted phenyl or optionally substituted heterocyclic aryl radical;
R2 represents H or phenylalkyl;
R4 represents H, quinolyl, 34-methylenedioxyphenyl or one of the phenyl or pyridyl radicals optionally substituted by a radical or radicals chosen in particular from alkyl, alkoxy, alkylthio, optionally protected hydroxy, amino, alkylamino, dialkylamino;
R5 represents H or an imidazolyl, phenyl, nitrophenyl, phenylalkyl radical, or also a —CO—N(R7)(R8) radical, in which R7 and R8 represent independently H, phenyl, phenylalkyl, alkyl or alkoxy;
or R4 and R5 in combination form a group of formula —CH═CH—CH═CH—;
Y is a phenylene radical substituted by a phenyl, phenoxy or phenylalkoxy radical, or a group of formula —CH(R3)-, in which R3 represents H or a radical of formula —(CH₂)ₙ—R6, in which R6 represents an optionally protected hydroxy, acyl, carboxy, acylamino, alkoxy, phenylalkoxy, alkylthio, optionally substituted phenyl, optionally substituted pyridyl, pyrazinyl, pyrimidinyl, furyl, imidazolyl, naphthyl, N-alkylindolyl or 3,4-methylenedioxyphenyl radical and n is an integer from 0 to 3;
R2 and R3 taken together with the carbon atoms which carry them can form a phenyl group;
X represents S or NR9;
R9 representing H, an alkyl or cycloalkyl radical, or also a benzyl radical optionally substituted once on its phenyl part by H, alkyl or alkoxy;
are inhibitors of the NO synthases and can be used to treat diseases which include in particular cardiovascular or cerebral ischemia, cerebral hemorrhage, disorders of the central nervous system, Alzheimer's disease, multiple sclerosis, diabetes, hepatitis, migraine, rheumatoid arthritis and osteoporosis.

The Applicant has also described in the PCT Patent Application WO 01/26656 a family of compounds comprising derivatives of 4-phenylthiazole and 4-phenylimidazole. The compounds of this family inhibit monoamine oxydases (MAO) and/or lipidic peroxidation and/or act as modulators of the sodium channels.

The Applicant has now unexpectedly discovered that certain particular compounds belonging to the family described in the PCT Patent Application WO 01/26656, namely the compounds described in the examples of the present application, possess particularly advantageous properties compared to their closest homologues of the PCT Patent Application WO 01/26656.

These advantageous properties make these compounds particularly suitable for a use in the treatment of neurodegenerative diseases, and in particular those indicated previously, pain particularly pain of neuropathic origin) or epilepsy.

The invention therefore relates to the use of one of the following compounds:
butyl 2-[4-(4-aminophenyl)-1H-imidazol-2-yl]ethylcarbamate;
N,2-dimethyl-1-[4-(10H-phenothiazin-2-yl)-1,3-thiazol-2-yl]propan-1-amine;
N,2-dimethyl-1-[4-(10H-phenoxazin-2-yl)-1,3-thiazol-2-yl]propan-1-amine;
N,3-dimethyl-1-[4-(10H-phenoxazin-2-yl)-1,3-thiazol-2-yl]butan-1-amine;
N,3-dimethyl-1-[4-(10H-phenothiazin-2-yl)-1,3-thiazol-2-yl]butan-1-amine;
2,6-di-tert-butyl-4-{2-[3-methyl-1-(methylamino)butyl]-1,3-thiazol-4-yl}phenol;
[4-(3,5-d-tert-butylphenyl)-1,3-thiazol-2-yl]methylamine;
2,6-di-tert-butyl-4-{2-[(1S)-1-(methylamino)ethyl]-1,3-thiazol-4-yl}phenol;
2,6-di-tert-butyl-4-{2-[(1R)-1-(methylamino)ethyl]-1,3-thiazol-4-yl}phenol;
N-{[4-(3,5-di-tert-butylphenyl)-1,3-thiazol-2-yl]methyl}-N-methylamine;
N-methyl-N-{[4-(3,4,5-trimethoxyphenyl)-1,3-thiazol-2-yl]methyl}amine;
ethyl N-{[4-(3,5-di-tert-butyl-4-hydroxyphenyl)-1,3-thiazol-2-yl]methyl}glycinate;
N-{[4-(3,5-di-tert-butyl-4-hydroxyphenyl)-1,3-thiazol-2-yl]methyl}glycine;
2,6-di-tert-butyl-4-{2-[(4-methoxypiperidin-1-yl)methyl]-1,3-thiazol-4-yl phenol;
N-methyl-N-{(1S)-2-methyl-1-[4-(10H-phenothiazin-2-yl)-1,3-thiazol-2-yl]propyl}amine;
N,2-dimethyl-1-[4-(10-methyl-10H-phenothiazin-2-yl)-1,3-thiazol-2-yl]propan-1-amine;
N-methyl-N-{(S)-2-methyl-1-[4-(10H-phenoxazin-2-yl)-1,3-thiazol-2-yl]propyl}amine;
4-{2-[(1R)-1-aminoethyl]-1,3-thiazol-4-yl}-2,6-di-tert-butylphenol;
4-{2-[(1S)-1-aminoethyl]-1,3-thiazol-4-yl}-2,6-di-tert-butylphenol;
4-[2-(1-aminocyclopropyl)-1,3-thiazol-4-yl]-2,6-di-tert-butylphenol;
4-{2-[(methylamino)methyl]-1,3-thiazol-4-yl}benzene-1,2-diol;
N-methyl-N-{(1R)-2-methyl-1-[4-(10H-phenothiazin-2-yl)-1,3-thiazol-2-yl]propyl}amine;
(1R)-2-methyl-1-[4-(10H-phenothizin-2-yl)-1,3-thiazol-2-yl]propan-1-amine;
N-methyl-N-{(1R)-2-methyl-1-[4-(10H-phenoxazin-2-yl)-1,3-thiazol-2-yl]propyl}amine;
N²-{[4-(3,5-di-tert-butyl-4-hydroxyphenyl)-1,3-thiazol-2-yl]methyl}glycinamide;
ethyl N-{[4-(3,5-di-tert-butyl-4-hydroxyphenyl)-1,3-thiazol-2-yl]methyl}-N-(2-ethoxy-2-oxoethyl)glycinate;
4-(3,5-di-tert-butyl-4-methoxyphenyl)-2-(methoxymethyl)-1,3-thiazole;
4-[2-(1-aminocyclopentyl)-1,3-thiazol-4-yl]-2,6-di-tert-butylphenol;
   or a pharmaceutically acceptable salt of one of the latter; for preparing a medicament intended to inhibit monoamine oxydases (MAO) and/or lipidic peroxidation and/or to act as modulators of the sodium channels.

The invention also relates to the use of one of the abovementioned compounds or pharmaceutically acceptable salts for preparing a medicament intended to treat disorders/pathologies chosen from neurodegenerative diseases, pain (particularly pain of neuropathic origin) and epilepsy.

Preferably, the neurodegenerative diseases intended to be treated by a medicament prepared according to the invention will be chosen from Parkinson's disease, Alzheimer's disease, Huntington's chorea and amyotrophic lateral sclerosis.

According to a first variant of the invention, the compounds of Examples 2 to 28 (sometimes described in the form of salts) or their pharmaceutically acceptable salts are preferred when an inhibitory activity on MAO's and/or the ROS's is sought in the first place. More preferentially, the compounds of Examples 6, 7, 10, 15 to 17, 22 and 28 (described in the form of hydrochloride salts), or their pharmaceutically acceptable salts, will be used for preparing a medicament according to the invention when an inhibitory activity on MAO's and/or the ROS's is sought in the first place. Even more preferentially, the compound of Example 28 (described in the form of its hydrochloride salt), or its pharmaceutically acceptable salts, will be used for preparing a medicament according to the invention when an inhibitory activity on MAO's and/or the ROS's is sought in the first place.

According to another variant of the invention, the compounds of Examples 1, 8 to 10 and 25 (sometimes described in the form of salts), or their pharmaceutically acceptable salts, are preferred when a modulating activity on the sodium channels is sought in the first place. More preferentially, the compound of Example 1, or its pharmaceutically acceptable salts, will be preferred for preparing a medicament according to the invention when a modulating activity on the sodium channels is sought in the first place.

A subject of the invention is also, as medicaments, the following compounds:
butyl 2-[4-(4-aminophenyl)-1H-imidazol-2-yl]ethylcarbamate;
N,2-dimethyl-1-[4-(10H-phenothiazin-2-yl)-1,3-thiazol-2-yl]propan-1-amine;
N,2-dimethyl-1-[4-(10H-phenoxazin-2-yl)-1,3-thiazol-2-yl]propan-1-amine;
N,3-dimethyl-1 [4-(10H-phenoxazin-2-yl)-1,3-thiazol-2-yl]butan-1-amine;
N,3-dimethyl-1-[4-(10H-phenothiazin-2-yl)-1,3-thiazol-2-yl]butan-1-amine;
2,6-di-tert-butyl-4-{2-[3-methyl-1-(methylamino)butyl]-1,3-thiazol-4-yl}phenol;
[4-(3,5-di-tert-butylphenyl)-1,3-thiazol-2-yl]methylamine;
2,6-di-tert-butyl-4-{2-[(1S)-1-(methylamino)ethyl]-1,3-thiazol-4-yl}phenol;
2,6-di-tert-butyl-4-{2-[(1R)-1-(methylamino)ethyl]-1,3-thiazol-4-yl}phenol;
N-{[4-(3,5-di-tert-butylphenyl)-1,3-thiazol-2-yl]methyl}-N-methylamine;
N-methyl-N-{[4-(3,4,5-trimethoxyphenyl)-1,3-thiazol-2-yl]methyl}amine;
ethyl N-{[4-(3,5-di-tert-butyl-4-hydroxyphenyl)-1,3-thiazol-2-yl]methyl}glycinate;
N-{[4-(3,5-di-tert-butyl-4-hydroxyphenyl)-1,3-thiazol-2-yl]methyl}glycine;
2,6-di-tert-butyl-4-{2-[(4-methoxypiperidin-1-yl)methyl]-1,3-thiazol-4-yl phenol;
N-methyl-N-{(1S)-2-methyl-1-[4-(10H-phenothiazin-2-yl)-1,3-thiazol-2-yl]propyl}amine;
N,2-dimethyl-1-[4-(10-methyl-10H-phenothiazin-2-yl)-1,3-thiazol-2-yl]propan-1-amine;
N-methyl-N-{(1S)-2-methyl-1-[4-(10H-phenoxazin-2-yl)-1,3-thiazol-2-yl]propyl}amine;
4-{2-[(1R)-1-aminoethyl]-1,3-thiazol-yl}-2,6-di-tert-butylphenol;
4-{2-[(1S)-1-aminoethyl]-1,3-thiazol-4-yl}-2,6-di-tert-butylphenol;
4-[2-(1-aminocyclopropyl)-1,3-thiazol-4-yl]-2,6-di-tert-butylphenol;
4-{2-[(methylamino)methyl]-1,3-thiazol-4-yl}benzene-1,2-diol;
N-methyl-N-{(1R)-2-methyl-1-[4-(10H-phenothiazin-2-yl)-1,3-thiazol-2-yl]propyl}amine;
(1R)-2-methyl-1-[4-(10H-phenothiazin-2-yl)-1,3-thiazol-2-yl]propan-1-amine;
N-methyl-N-{(1R)-2-methyl-1-[4-(10H-phenoxazin-2-yl)-1,3-thiazol-2-yl]propyl}amine;
N²-{[4-(3,5-di-tert-butyl-4-hydroxyphenyl)-1,3-thiazol-2-yl]methyl}glycinamide;
ethyl N-{[4-(3,5-di-tert-butyl-4-hydroxyphenyl)-1,3-thiazol-2-yl]methyl}-N-(2-ethoxy-2-oxoethyl)glycinate;
4-(3,5-di-tert-butyl-4-methoxyphenyl)-2-(methoxymethyl)-1,3-thiazole;
4-[2-(1-aminocyclopentyl)-1,3-thiazol-4-yl]-2,6-di-tert-butylphenol;
   and pharmaceutically acceptable salts of the latter.

The invention also relates to the pharmaceutical compositions containing, as active ingredient, at least one of the following compounds
butyl 2-[4-(4-aminophenyl)-1H-imidazol-2-yl]ethylcarbamate;
N,2-dimethyl-1-[4-(10H-phenothiazin-2-yl)-1,3-thiazol-2-yl]propan-1-amine;
N,2-dimethyl-1-[4-(10H-phenoxazin-2-yl)-1,3-thiazol-2-yl]propan-1-amine;
N,3-dimethyl-1-[4-(10H-phenoxazin-2-yl)-1,3-thiazol-2-yl]butan-1-amine;
N,3-dimethyl-1-[4-(10H-phenothiazin-2-yl)-1,3-thiazol-2-yl]butan-1-amine;
2,6-di-tert-butyl-4-{2-[3-methyl-1-(methylamino)butyl]-1,3-thiazol-4-yl}phenol;
[4-(3,5-di-tert-butylphenyl)-1,3-thiazol-2-yl]methylamine;
2,6-di-tert-butyl-4-{2-[(1S)-1-(methylamino)ethyl]-1,3-thiazol-4-yl}phenol;
2,6-di-tert-butyl-4-{2-[(1R)-1-(methylamino)ethyl]-1,3-thiazol-4-yl}phenol;
N-{[4-(3,5-di-tert-butylphenyl)-1,3-thiazol-2-yl]methyl}-N-methylamine;
N-methyl-N-{[4-(3,4,5-trimethoxyphenyl)-1,3-thiazol-2-yl]methyl}amine;
ethyl N-{[4-(3,5-di-tert-butyl-4-hydroxyphenyl)-1,3-thiazol-2-yl]methyl}glycinate;
N-{[4-(3,5-di-tert-butyl-4-hydroxyphenyl)-1,3-thiazol-2-yl]methyl}glycine;
2,6-di-tert-butyl-4-{2-[(4-methoxypiperidin-1-yl)methyl]-1,3-thiazol-4-yl}phenol;
N-methyl-N-{(1S)-2-methyl-1-[4-(10H-phenothiazin-2-yl)-1,3-thiazol-2-yl]propyl}amine;
N,2-dimethyl-1-[4-(10-methyl-10H-phenothiazin-2-yl)-1,3-thiazol-2-yl]propan-1-amine;
N-methyl-N-{(1S)-2-methyl-1-[4-(10H-phenoxazin-2-yl)-1,3-thiazol-2-yl]propyl}amine;
4-{2-[(1R)-1-aminoethyl]-1,3-thiazol-4-yl}-2,6-di-tert-butylphenol;
4-{2-[(1S)-1-aminoethyl]-1,3-thiazol-4-yl}-2,6-di-tert-butylphenol;
4-[2-(1-aminocyclopropyl)-1,3-thiazol-4-yl]-2,6-di-tert-butylphenol;
4 {2-[(methylamino)methyl]-1,3-thiazol-4-yl benzene-1,2-diol;
N-methyl-N-{(1R)-2-methyl-1-[4-(10H-phenothiazin-2-yl)-1,3-thiazol-2-yl]propyl}amine;
(1R)-2-methyl-1-[4-(10H-phenothiazin-2-yl)-1,3-thiazol-2-yl]propan-1-anine;
N-methyl-N-{(1R)-2-methyl-1-[4-(10H-phenoxazin-2-yl)-1,3-thiazol-2-yl]propyl}amine;
N²-{[4-(3,5-di-tert-butyl-4-hydroxyphenyl)-1,3-thiazol-2-yl]methyl}glycinamide;
ethyl N-{[4-(3,5-di-tert-butyl-4-hydroxyphenyl)-1,3-thiazol-2-yl]methyl}-N-(2-ethoxy-2-oxoethyl)glycinate;
4-(3,5-di-tert-butyl-4-methoxyphenyl)-2-(methoxymethyl)-1,3-thiazole;
4-[2-(1-aminocyclopentyl)-1,3-thiazol-4-yl]-2,6-di-tert-butylphenol;
   or a pharmaceutically acceptable salt of one of these compounds

The invention also relates to the following compounds as new industrial products:
butyl 2-[4-(4-aminophenyl)-1H-imidazol-2-yl]ethylcarbamate;
N,2-dimethyl-1-[4-(10H-phenothiazin-2-yl)-1,3-thiazol-2-yl]propan-1-amine;
N,2-dimethyl-1-[4-(10H-phenoxazin-2-yl)-1,3-thiazol-2-yl]propan-1-amine;
N,3-dimethyl-1-[4-(10H-phenoxazin-2-yl)-1,3-thiazol-2-yl]butan-1-amine;
N,3-dimethyl-1-[4-(10H-phenothiazin-2-yl)-1,3-thiazol-2-yl]butan-1-amine;
2,6-di-tert-butyl-4-{2-[3-methyl-1-(methylamino)butyl]-1,3-thiazol-4-yl}phenol;
[4-(3,5-di-tert-butylphenyl)-1,3-thiazol-2-yl]methylamine;
2,6-di-tert-butyl-4-{2-[(1S)-1-(methylamino)ethyl]-1,3-thiazol-4-yl}phenol;
2,6-di-tert-butyl-4-{2-[(R)-1-(methylamino)ethyl]-1,3-thiazol-4-yl}phenol;
N-{[4-(3,5-di-tert-butylphenyl)-1,3-thiazol-2-yl]methyl}-N-methylamine;
N-methyl-N-{[4-(3,4,5-trimethoxyphenyl)-1,3-thiazol-2-yl]methyl}amine;
ethyl N-{[4-(3,5-di-tert-butyl-4-hydroxyphenyl)-1,3-thiazol-2-yl]methyl}glycinate;
N-{[4-(3,5-di-tert-butyl-4-hydroxyphenyl)-1,3-thiazol-2-yl]methyl}glycine;
2,6-di-tert-butyl-4-{2-[(4-methoxypiperidin-1-yl)methyl]-1,3-thiazol-4-yl}phenol;
N-methyl-N-{(1S)-2-methyl-1-[4-(10H-phenothiazin-2-yl)-1,3-thiazol-2-yl]propyl}amine;
N,2-dimethyl-1-[4-(10-methyl-10H-phenothiazin-2-yl)-1,3-thiazol-2-yl]propan-1-amine;
N-methyl-N-{(1S)-2-methyl-1-[4-(10H-phenoxazin-2-yl)-1,3-thiazol-2-yl]propyl}amine;
4-{2-[(1R)-1-aminoethyl]-1,3-thiazol-4-yl}-2,6-di-tert-butylphenol;
4-{2-[(JS)-1-aminoethyl]-1,3-thiazol-4-yl}-2,6-di-tert-butylphenol;
4-[2-(1-aminocyclopropyl)-1,3-thiazol-4-yl]-2,6-di-tert-butylphenol;
4-{2-[(methylamino)methyl]-1,3-thiazol-4-yl}benzene-1,2-diol;
N-methyl-N-{(JR)-2-methyl-1-[4-(10H-phenothiazin-2-yl)-1,3-thiazol-2-yl]propyl}amine;
(1R)-2-methyl-1-[4-(10H-phenothiazin-2-yl)-1,3-thiazol-2-yl]propan-1-amine;
N-methyl-N-{(1R)-2-methyl-1-[4-(10H-phenoxazin-2-yl)-1,3-thiazol-2-yl]propyl}amine;
N²-{[4-(3,5-di-tert-butyl-4-hydroxyphenyl)-1,3-thiazol-2-yl]methyl}glycinamide;
ethyl N-{[4-(3,5-di-tert-butyl-4-hydroxyphenyl)-1,3-thiazol-2-yl]methyl}-N-(2-ethoxy-2-oxoethyl)glycinate;
4-(3,5-di-tert-butyl-4-methoxyphenyl)-2-(methoxymethyl)-1,3-thiazole;
4-[2-(1-aminocyclopentyl)-1,3-thiazol-4-yl]-2,6-di-tert-butylphenol;
   and salts of these compounds.

In certain cases, the compounds according to the present invention can contain asymmetrical carbon atoms. As a result, the compounds according to the present invention have two possible enantiomeric forms, i.e. the “R” and “S” configurations. The present invention includes the two enantiomeric forms and all combinations of these forms, including the racemic “RS” mixtures. For the sake of simplicity, when no specific configuration is indicated in the structural formulae or names, it should be understood that the two enantiomeric forms and their mixtures are represented

By salt is meant, in particular, in the present application addition salts with organic or inorganic acids as well as the salts formed using bases.

By pharmaceutically acceptable salt, is meant in particular the addition salts with inorganic acids such as hydrochloride, hydrobromide, hydroiodide, sulphate, phosphate, diphosphate and nitrate or with organic acids such as acetate, maleate, fumarate, tartrate, succinate, citrate, lactate, methanesulphonate, p-toluenesulphonate, pamoate and stearate. Also included in the field of the present invention, when they can be used, are the salts formed from bases such as sodium or potassium hydroxide. For other examples of pharmaceutically acceptable salts, reference can be made to “Salt selection for basic drugs”, Int. J. Pharm. (1986), 33, 201-217.

The pharmaceutical compositions according to the present invention can be in the form of solids, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions containing a compound of the invention can also be presented in liquid form, for example, solutions, emulsions, suspensions or syrups. Appropriate liquid supports can be, for example, water, organic solvents such as glycerol or glycols, similarly their mixtures, in varying proportions, in water.

The administration of a medicament according to the invention can be done by topical, oral, parenteral route, by intramuscular injection, etc.

The administration dose envisaged for a medicament according to the invention is comprised between 0.1 mg to 10 g according to the type of active compound used.

In accordance with the invention, the compounds of the invention can be prepared by the processes described below.

Preparation of the Compounds of the Invention:

The compounds of the present invention can all be prepared according to techniques described in PCT publication WO 01/26656.

Unless defined otherwise, all the technical and scientific terms used here have the same meaning as that usually understood by an ordinary specialist in the field to which this invention belongs. Likewise, all publications, patent applications, all patents and all other references mentioned here are incorporated by way of reference.

The following examples are presented to illustrate the above procedures and must in no case be considered as limiting the scope of the invention.

### EXAMPLES

### Preparation 1

### 4-[3,5-bis(1,1-dimethylethyl)-4-hydroxyphenyl]-N-methyl-2-thiazolemethanamine

### Stage 1

### N-Boc-sarcosinamide

15.0 g (0.120 mol) of sarcosinamide hydrochloride (N-Me-Gly-NH₂.HCl) is dissolved in dichloromethane containing 46.2 ml (0.265 mol) of diisopropylethylamine. The mixture is cooled down to 0° C. then Boc-O-Boc (28.8 g; 0.132 mol) is added in fractions and the mixture is stirred overnight at ambient temperature. The reaction medium is then poured into ice-cooled water followed by extraction with dichloromethane. The organic phase is washed successively with a 10% aqueous solution of sodium bicarbonate and with water, then finally with a saturated solution of sodium chloride. The organic phase is then dried over magnesium sulphate, filtered and concentrated under vacuum. The product obtained is purified by crystallization from diisopropyl ether in order to produce a white solid with a yield of 72%. Melting point; 103° C.

### Stage 2

### 2-{[(1,1-dimethylethoxy)carbonyl]methyl}amino-ethanethioamide

16.0 g (0.085 mol) of the intermediate obtained in Stage 1 is dissolved in dimethoxyethane (500 ml) and the solution obtained is cooled down to 5° C. Sodium bicarbonate (28.5 g; 0.34 mol) then, in small portions, (P₂S₅)₂ (38.76 g; 0.17 mol) are added. The reaction medium is allowed to return to ambient temperature under stirring over 24 hours. After evaporation of the solvents under vacuum, a 10% aqueous solution of sodium bicarbonate is added to the residue and the solution is extracted using ethyl acetate. The organic phase is washed successively with a 10% aqueous solution of sodium bicarbonate and with water, then finally with a saturated solution of sodium chloride. The organic phase is then dried over magnesium sulphate, filtered and concentrated under vacuum. The product obtained is purified by crystallization from ether in order to produce a white solid with a yield of 65%. Melting point: 150-151° C.

### Stage 3

### 4-[3,5-bis(1,1-dimethylethyl)-4-hydroxyphenyl]-N-[(1,1-dimethylethoxy)-carbonyl]-N-methyl-2-thiazolemethanamine

The intermediate obtained in Stage 2 (4.3 g; 2.11 mmol) and bromo-1-(3,5-ditert-butyl-4-hydroxyphenyl)ethanone (6.9 g; 2.11 mmol) are dissolved in benzene (75 ml) under an argon atmosphere, then the mixture is stirred at ambient temperature for 12 hours. The reaction medium is heated under reflux for 4 hours. After evaporation of the solvents, the residue is diluted with dichloromethane and washed with a saturated solution of NaCl. The organic phase is separated, dried over magnesium sulphate, filtered and concentrated under vacuum. The expected product is obtained after chromatography on a silica column (eluent: 20% ethyl acetate in heptane) in the form of an oil which crystallizes very slowly in a refrigerator with a yield of 28%. Melting point: 126.5-127.3° C.

### Stage 4

### 4-[3,5-bis(1,1-dimethylethyl)-4-hydroxyphenyl]-N-methyl-2-thiazolemethanamine

2.3 ml (29 mmol) of trifluoroacetic acid is added dropwise, at 0 CC to a solution of 2.5 g (5.8 mmol) of the intermediate obtained in Stage 3 and 2 ml (1.6 mmol) of triethylsilane in 50 ml of dichloromethane. After stirring for one hour, the reaction mixture is concentrated under vacuum and the residue is diluted in 100 ml of ethyl acetate and 50 ml of a saturated solution of NaHCO₃. After stirring and decantation, the organic phase is dried over magnesium sulphate, filtered and concentrated under vacuum. The residue is taken up in heptane in order to produce, after drying, a white solid with a yield of 73%. Melting point: 136° C.

### Stage 5

### 4-[3,5-bis(1,1-dimethylethyl)-4-hydroxyphenyl]-N-methyl-2-thiazolemethanamine hydrochloride

2.0 g (0.602 mmol) of the intermediate obtained in Stage 4 is dissolved in anhydrous ether. The solution is cooled down to 0° C. then 18 ml (1.81 mmol) of a 1N solution of HCl in ether is added dropwise. The mixture is allowed to return to ambient temperature under stirring. After filtering and drying under vacuum, a white solid is obtained with a yield of 92%. Melting point: 185.3-186.0° C.

### Preparation 2

### 2,6-di(tert-butyl)-4-(2-{[methyl(2-propynyl)amino]methyl}-1,3-thiazol-4-yl)phenol

0.52 ml (3.7 mmol) of triethylamine and an excess of 0.56 g (7.5 mmol) of chloropropargyl are added dropwise at 0° C. to a solution of 0.5 g (1.5 mmol) of the compound of Preparation 1 in 15 ml of acetonitrile. After stirring overnight, the reaction mixture is concentrated under vacuum and the residue is diluted with dichloromethane and 50 ml of a saturated solution of NaCl. After stirring and decantation, the organic phase is separated and dried over magnesium sulphate, filtered and concentrated under vacuum. The expected product is obtained after chromatography on a silica column (eluent: 20% ethyl acetate in heptane). After evaporation, the pure fractions produce a white solid with a yield of 20%. Melting point: 210-215° C.

MH+=371.20.

### Preparation 3

### benzyl {4-[3,5-di(tert-butyl)-4-hydroxyphenyl]-1,3-thiazol-2-yl}methylcarbamate

The compound is prepared according to an experimental protocol described in the Patent Application WO 98/58934 (see preparation of intermediates 26.1 and 26.2), using Z-Gly-NH₂ in place of the N-Boc sarcosinamide. The expected compound is obtained in the form of a pale yellow oil with a yield of 99%.

MH+=453.20.

### Preparation 4

### 4-[2-(aminomethyl)-1,3-thiazol-4-yl]-2,6-di(tert-butyl)phenol

0.1 ml of a 40% solution of potassium hydroxide is added dropwise to a solution of 0.106 g (1.1 mmol) of the compound of Preparation 3 in 10 ml of methanol. After overnight stirring under reflux, the reaction mixture is concentrated under vacuum and 0.15 the residue is diluted with dichloromethane and washed with a 1N solution of HCl then with 50 ml of a saturated solution of NaCl. The organic phase is separated and dried over magnesium sulphate, filtered and concentrated under vacuum. The expected product is obtained after chromatography on a silica column (eluent: 5% ethanol in dichloromethane) in the form of a brown foam with a yield of 76%.

MH+=319.29.

### Preparation 5

### 2-[3,5-bis(1,1-dimethylethyl)-4-hydroxyphenyl]4-oxazoleethanol

[this is intermediate 1C of the PCT Application WO 99/09829; alternatively, this compound can also be obtained according to the procedure described in J. Med. Chem. (1996), 39, 237-245.]

### Preparation 6

### 2,6-ditert-butyl-4-(4-{2-[methyl(2-propynyl)amino]ethyl}-1,3-oxazol-2-yl)phenol

The compound of Preparation 5 is convened to a brominated derivative, intermediate 3, according to the procedure indicated in Diagram 1 (c) of the PCT Application WO 99/09829. Then the brominated derivative (0.5 g; 1.31 mmol) is added to a solution of N-methylpropargylamine 0.34 ml (3.94 mmol) and potassium carbonate (1.11 g) in dimethylformamide (20 ml). After overnight stirring at 80° C., the reaction mixture is concentrated under vacuum and the residue is diluted with dichloromethane and 50 ml of a saturated solution of NaCl. After stirring and decantation, the organic phase is separated and dried over magnesium sulphate, filtered and concentrated under vacuum. The expected product is obtained after chromatography on a silica column (eluent: 50% ethyl acetate in heptane). After evaporation, the pure fractions produce a yellow oil with a yield of 24%.

MH+=369.30.

### Preparation 7

### 2,6-ditert-butyl-4-{4-[2-(1-piperazinyl)ethyl]-1,3-oxazol-2-yl}phenol hydrochloride

### Stage 1

### tert-butyl 4-{2-[2-(3,5-ditert-butyl-4-hydroxyphenyl)-1,3-oxazol-4-yl]ethyl}-1-piperazinecarboxylate

The experimental protocol used is identical to that described for Preparation 6, tert-butyl piperazinecarboxylate being used as starting product in place of the N-methylpropargylamine. A brown oil is obtained with a yield of 72%.

MH+=486.20.

### Stage 2

### 2,6-ditert-butyl-4-{4-[2-(1-piperazinyl)ethyl]-1,3-oxazol-2-yl}phenol hydrochloride

A stream of HCl gas is passed bubblewise into a solution at 0° C. of the intermediate obtained in Stage 1 (0.450 g; 9.27 mmol) in ethyl acetate (30 ml). The mixture is left to return to ambient temperature overnight. A stream of argon is passed through the reaction mass, then the powder obtained is filtered and washed with ethyl acetate then with ether in order to produce a white solid with a yield of 70%. Melting point: >200° C.

### Preparation 8

### N-methyl-[4-(10H-phenothiazin-2-yl)-1,3-thiazol-2-yl]methanamine hydrochloride

### Stage 1

### 2-chloro-1-(10H-phenothiazin-2-yl)ethanone

2-bromo-1-[10-(chloroacetyl)-10H-phenothiazin-2-yl)ethanone (2.2 g; 5.55 mmol; prepared according to a protocol described in J. Heterocyclic. Chem. (1978), 15, 175, followed by a Friedel-Crafts reaction) is dissolved hot in a mixture of acetic acid (20 ml) and 20% HCl (5.5 ml) and the mixture obtained is heated under reflux for 30 minutes. The reaction mixture is allowed to cool down, the precipitate is filtered, the mixture rinsed with acetic acid (5 ml) and dried under vacuum, the solid obtained is purified by crystallization from toluene in order to produce a brown product with a yield of 82%. Melting point: 190-191° C. (value in the literature: 197-198° C.).

### Stage 2

### N-methyl-[4-(10H-phenothiazin-2-yl)-1,3-thiazol-2-yl]methanamine hydrochloride

The intermediate obtained in Stage 1 (0.280 g; 1.0 mmol) and tert-butyl 2-amino-2-thioxoethyl(methyl)carbamate (0.204 g; 1.0 mmol; described for example in PCT Patent Application WO 98/58934) are dissolved in toluene and the mixture is heated under reflux for 18 hours. After the toluene is evaporated off and the reaction mixture cooled down to 0° C., the latter is taken up in a 4N solution of HCl in dioxane (10 ml) and the mixture stirred for one hour at 0° C. before allowing the temperature to return to ambient temperature. The solid formed is filtered and rinsed with ether. The expected product is obtained after purification by crystallization from hot acetic acid in order to obtain a greenish solid, Melting point: >275° C.

### Preparation 9

### butyl 2-(4-[1,1′-biphenyl]-4-yl-1H-imidazol-2-yl)ethylcarbamate

### Stage 1

### N-(butoxycarbonyl)-β-alanine

A solution containing β-alanine (8.9 g; 0.1 mol) and 100 ml of a 1N solution of sodium hydroxide is cooled down to 10° C. n-butyl chloroformate (13.66 g; 0.1 mol) and 50 ml of a 2N solution of sodium hydroxide are added simultaneously. After stirring for 16 hours at 23° C., approximately 10 ml of a solution of concentrated hydrochloric acid (approximately 11 N) is added in order to adjust the pH to 4-5. The oil obtained is extracted with ethyl acetate (2×50 ml), washed with water then dried over magnesium sulphate. The product crystallizes from isopentane in the form of a white powder (yield of 68%). Melting point: 50.5° C.

### Stage 2

### butyl 2-(4-[1,1′-biphenyl]-4-yl-1H-imidazol-2-yl)ethylcarbamate

A mixture of N-butoxycarbonyl)-β-alanine prepared in Stage 1; 5.67 g; 0.03 mol) and caesium carbonate (4.89 g; 0.015 mol) in 100 ml of ethanol is stirred at 23 CC for 1 hour. The ethanol is eliminated by evaporation under reduced pressure in a rotary evaporator. The mixture obtained is dissolved in 100 ml of dimethylformamide then 4-phenyl-bromoacetophenone (8.26 g; 0.03 mol) is added. After stirring for 16 hours, the solvent is evaporated off under reduced pressure. The mixture obtained is taken up in ethyl acetate then the caesium bromide is filtered. The ethyl acetate of the filtrate is evaporated and the reaction oil is taken up in a mixture of xylene (100 ml) and ammonium acetate (46.2 g; 0.6 mol). The reaction medium is heated at reflux for approximately one hour and 30 minutes then, after cooling down, a mixture of ice-cooled water and ethyl acetate is poured into the reaction medium. After decantation, the organic phase is washed with a saturated solution of sodium bicarbonate, dried over magnesium sulphate then evaporated under vacuum. The solid obtained is filtered then washed with ether in order to produce a light beige-coloured powder (yield of 50%). Melting point: 136.7° C.

MH+=364.3.

### Preparation 10

### 2,5,7,8-tetramethyl-2-{2-[(methylamino)methyl]-1,3-thiazol-4-yl}-6-chromanol hydrochloride

### Stage 1

### 6-hydroxy-methoxy-N,2,5,7,8-pentamethyl-2-chromanecarboxamide

2.2 g (22.0 mmol) of O,N-dimethylhydroxylamine hydrochloride, triethylamine (6.2 ml), 3.0 g (22.0 mmol) of hydroxybenzotriazole and 4.2 g (22.0 mmol) of 1-(3-dimethylaminopropyl)-3-ethyl-carbodiimide hydrochloride are added successively to a solution of 5.0 g (20.0 mmol) of (R,S) 6-hydroxy-2,5,7,8-tetramethyl-2-chromanecarboxylic acid (Trolox®) in 175 ml of DMF. After the reaction mixture is stirred overnight at 25° C., the mixture is diluted with ice-cooled water and stirring is maintained for another 30 minutes. The product is extracted using 3 times 100 ml of ethyl acetate. The organic solution is washed successively with a 10% aqueous solution of sodium bicarbonate, with water, with a 10% aqueous solution of citric acid and finally with a saturated solution of sodium chloride. The organic phase is then dried over magnesium sulphate, filtered and concentrated under vacuum. The product obtained is purified by crystallization from ether in order to produce a white-coloured solid with a yield of 63%. Melting point: 139-140° C.

MH+=294.

### Stage 2

### 1-(6-hydroxy-2,5,7,8-tetramethyl-3,4-dihydro-2H-chromen-2-yl)ethanone

A solution of methyllithium (1.6 M; 31.25 ml; 50.0 mmol) is added dropwise at a temperature of −30° C. to a solution of 2.93 g (10.0 mmol) of the intermediate obtained in Stage 1 in 100 ml of THF and the mixture is left under stirring for 1 hour at −10° C. The reaction medium is hydrolyzed with NH₄Cl in a saturated aqueous solution. The product is extracted using 3 times 150 ml of ethyl acetate. The organic phase is finally washed with sodium chloride in a saturated aqueous solution before being dried over magnesium sulphate, filtered and concentrated under vacuum. The product obtained is purified by crystallization from diisopropyl ether in order to produce a white solid with a yield of 80.7%. Melting point: 97-98° C.

MH+=248.

### Stage 3

### 2-bromo-1-(6-hydroxy-2,5,7,8-tetramethyl-3,4-dihydro-2H-chromen-2-yl)ethanone

The intermediate obtained in Stage 2 (0.777 g; 3.13 mmol) is dissolved in ethanol (25 ml) under a stream of argon. The solution is cooled down to 0° C. and bromine (0.18 ml; 4.20 mmol) is added in one go (see J. Am. Chem. Soc. (1999), 121, 24), then the mixture is stirred for 30 minutes allowing the temperature to rise to ambient temperature. The excess bromine is eliminated by bubbling through argon then the mixture is left under stirring for 2.5 hours. The ethanol is evaporated off and the product obtained is purified by crystallization from toluene. After filtering and washing with isopentane, a brown solid is obtained with a yield of 36%. Melting point: decomposition from 125° C.

MH+=326.

### Stage 4

### 2,5,7,8-tetramethyl-2-{2-[(methylamino)methyl]-1,3-thiazol-4-yl}-6-chromanol hydrochloride

The experimental protocol used is analogous to that described for Stage 2 of Preparation 8, the intermediate obtained in Stage 3 of the present Preparation being used as the starting product instead of the intermediate obtained in Stage 1 of Preparation 8, and benzene replacing the toluene as solvent, The product obtained is purified by crystallization from a minimum amount of dichloromethane in order to produce a white solid with a yield of 48%. Melting point: 153-155° C.

### Preparation 11

### 3,5-ditert-butyl-4′-{2-[(methylamino)methyl]-1,3-thiazol-4-yl}-1,1′-biphenyl-4-ol hydrochloride

### Stage 1

### 3′,5′-ditert-butyl-4′-hydroxy-1,1′-biphenyl-4-carboxylic acid

5.0 g (1.41 mmol) of ethyl 3′,5′-ditert-butyl-4′-hydroxy-1,1′-biphenyl-4-carboxylate (Chem. Lett. (1998), 9, 931-932) is dissolved in ethanol (25 ml). The solution is cooled down to 0° C. then a 1N solution of soda is added dropwise. After stirring overnight at ambient temperature, the reaction medium is heated under reflux in order to complete the reaction. After evaporation of the solvents and dilution of the residue with water, the mixture obtained is acidified with a 1N solution of HCl and extraction is carried out with dichloromethane. The organic phase is washed with sodium chloride in a saturated aqueous solution before being dried over magnesium sulphate, filtered and concentrated under vacuum. The product obtained is purified by crystallization from diisopropyl ether in order to produce a yellow-white solid with a yield of 47%. Melting point: >240° C.

### Stage 2

### 3′,5′-ditert-butyl-4′-hydroxy-N-methoxy-N-methyl-1,1′-biphenyl-4-carboxamide

The experimental protocol used is identical to that described for Stage 1 of Preparation 10, with the acid obtained in Stage 1 of the present Preparation replacing the Trolox® as starting product. A yellowish solid is obtained with a yield of 93%. Melting point: 175.6-177° C.

### Stage 3

### 1-(3′,5′-ditert-butyl-4′-hydroxy-1,1′-biphenyl-4-yl)ethanone

The experimental protocol used is identical to that described for Stage 2 of Preparation 10, the intermediate obtained in Stage 2 of the present Preparation replacing the intermediate obtained in Stage 1 of Preparation 10. A white solid is obtained with a yield of 74%. Melting point: 144-144.7° C.

### Stage 4

### 2-bromo-1-(3′,5′-ditert-butyl-4′-hydroxy-1,1′-biphenyl-4-yl)ethanone

The experimental protocol used is identical to that described for Stage 3 of Preparation 10, the intermediate obtained in Stage 3 of the present Preparation replacing intermediate obtained in Stage 2 of Preparation 10. A yellow-orange oil is obtained which is sufficiently pure to be used in the following stage (yield of 100%).

### Stage 5

### tert-butyl [4-(3′,5′-ditert-butyl-4′-hydroxy-1,1′-biphenyl-4-yl)-1,3-thiazol-2-yl]methyl(methyl)carbamate

This compound is prepared according to the experimental protocol described for Stage 3 of Preparation 1, using the intermediate obtained in Stage 4 of the present Preparation instead of bromo-1-(3,5-ditert-butyl-4-hydroxyphenyl)ethanone. The expected compound is obtained in the form of a colourless oil with a yield of 46%.

MH+=509.43.

### Stage 6

### 3,5-ditert-butyl-4′-{2-[(methylamino)methyl]-1,3-thiazol-4-yl}-1,1′-biphenyl-4-ol hydrochloride

0.230 g (0.452 mmol) of the intermediate obtained in Stage 5 of the present Preparation is dissolved in ethyl acetate (20 ml). HCl gas is bubbled through the solution previously obtained cooled down to 0° C. The stirred mixture is then allowed to return to ambient temperature. The solid formed is filtered and washed with ethyl acetate then with ether before being dried under vacuum. A white solid is obtained with a yield of 85%. Melting point: 220-221° C.

### Preparation 12

### 2,6-dimethoxy-4-{2-[(methylamino)methyl]-1,3-thiazol-4-yl}phenol hydrochloride

### Stage 1

### 4-acetyl-2,6-dimethoxyphenyl acetate

3.0 g (15.3 mmol) of 3,5-dimethoxy-4-hydroxyacetophenone is dissolved in dichloromethane (30 ml) and 2.53 g (18.3 mmol) of K₂CO₃ is added. Triethylamine (2.6 ml) is then added dropwise, The reaction medium is cooled down to 0° C. and acetyl chloride (1.31 ml; 18.3 mmol) is added. The mixture is stirred for 24 hours at ambient temperature then poured into ice-cooled water. After extraction with dichloromethane, the organic phase is washed with sodium chloride in a saturated aqueous solution before being dried over magnesium sulphate, filtered and concentrated under vacuum. The product obtained is purified by crystallization from ether in order to produce a white solid with a yield of 99%. Melting point: 145° C.

### Stage 2

### 4-(bromoacetyl)-2,6-dimethoxyphenyl acetate

The intermediate obtained in Stage 1 (0.850 g; 3.57 mmol) is solubilized in ethyl acetate then 1.35 g (6.07 mmol) of previously dried CuBr₂ is added. The mixture is heated under reflux for 2.5 hours before being left to return to ambient temperature. Vegetable black is added and the mixture is stirred for 10 minutes. After filtering and evaporating to dryness, the solid obtained is taken up in diisopropyl ether. After filtering, a grey solid is obtained with a yield of 75%. Melting point: 124.2-126.3° C.

### Stage 3

### 4-(2-{[(tert-butoxycarbonyl)(methyl)amino]methyl}-1,3-thiazol-4-yl)-2,6-dimethoxyphenyl acetate

This compound is prepared according to the experimental protocol described for Stage 3 of Preparation 1, using the intermediate obtained in Stage 2 of the present Preparation instead of bromo-1-(3,5-ditert-butyl-4-hydroxyphenyl)ethanone. The expected compound is obtained in the form of a white solid with a yield of 55%. Melting point: 135.2-137.4° C.

### Stage 4

### tert-butyl [4-(4-hydroxy-3,5-dimethoxyphenyl)-1,3-thiazol-2-yl]methyl(methyl)carbamate

0.530 g (1.25 mmol) of the intermediate obtained in Stage 3 is dissolved in methanol (20 ml). The solution is cooled down using an ice bath then a 1N solution of NaOH is added dropwise. The mixture is left to return to ambient temperature under stirring. After evaporation to dryness and dilution of the residue with water, the solution is neutralised using citric acid followed by extraction with dichloromethane. The organic phase is washed with sodium chloride in a saturated aqueous solution before being dried over magnesium sulphate, filtered and concentrated under vacuum. The product is obtained in the form of a yellow oil with a yield of 96%. MH+=381.20.

### Stage 5

### 2,6-dimethoxy-4-{2-[(methylamino)methyl]-1,3-thiazol-4-yl}phenol hydrochloride

The experimental protocol used is identical to that described for Stage 6 of Preparation 11, the intermediate obtained in Stage 4 of the present Preparation replacing the intermediate obtained am Stage 5 of Preparation 11. A light beige solid is obtained with a yield of 97%. Melting point: 229.8-232.0° C.

### Preparation 13

### 2,6-ditert-butyl-4-[2-(hydroxymethyl)-1,3-thiazol-4-yl]phenol

[this is intermediate 6.d₁) of Patent Application EP 432 740]

### Stage 1

### [4-(3,5-ditert-butyl-4-hydroxyphenyl)-1,3-thiazol-2-yl]methyl pivalate

This compound is prepared according to a protocol identical to that described for Stage 3 of Preparation 1, using 2-(tert-butylcarbonyloxy)thioacetamide instead of the 2-{[(1,1-dimethylethoxy)carbonyl]methyl}amino-ethanethioamide and toluene replacing the benzene. The expected compound is obtained in the form of a white solid with a yield of 100%. Melting point 114.6-116.0° C.

### Stage 2

### 2,6-ditert-butyl-4-[2-(hydroxymethyl)-1,3-thiazol-4-yl]phenol

The experimental protocol used is identical to that described for Stage 4 of Preparation 12, the intermediate obtained in Stage 1 of the present Preparation replacing the intermediate obtained in Stage 3 of Preparation 12. A white solid is obtained with a yield of 88%. Melting point: 126.4-127.4° C.

### Preparation 14

### 2,6-ditert-butyl-4-{2-[2-(methylamino)ethyl]-1,3-thiazol-4-yl}phenol hydrochloride

### Stage 1

### tert-butyl 2-cyanoethyl(methyl)carbamate

0.1 mol of N-methyl-β-alaninenitrile is dissolved in dichloromethane (100 ml) containing 20.9 ml (0.12 mol) of diisopropylethylamine. The mixture is then cooled down to 0° C. then Boc-O-Boc (26.2 g; 0.12 mol) is added by fractions, then the mixture is stirred overnight at ambient temperature. The reaction medium is then poured into ice-cold water and extracted with dichloromethane. The organic phase is washed successively with a 10% aqueous solution of sodium bicarbonate and with water, then finally with a saturated solution of sodium chloride. The organic phase is then dried over magnesium sulphate, filtered and concentrated under vacuum. The reddish brown oil obtained is used as it is in the following stage.

### Stage 2

### tert-butyl 3-amino-3-thioxopropyl(methyl)carbamate

43.4 mmol of the intermediate obtained in Stage 1 is dissolved in ethanol (40 ml) containing triethylamine (6.1 ml). H₂S is then bubbled through the mixture for 3 hours before evaporating the solvents to dryness. The expected product is obtained after chromatography on a silica column (eluent: 50% ethyl acetate in heptane) in the form of a light orange oil. Crystallization of this oil from diisopropyl ether gives a white solid with a yield of 15%. Melting point: 104° C.

### Stage 3

### 4-[3,5-bis(1,1-dimethylethyl)-4-hydroxyphenyl]-N-[(1,1-dimethylethoxy)-carbonyl]-N-methyl-2-thiazole ethanamine

The intermediate obtained in Stage 2 (2.11 mmol) and bromo-1-(3,5-ditert-butyl-4-hydroxyphenyl)ethanone (6.9 g; 2.11 mmol) are dissolved in toluene (75 ml) under an argon atmosphere then the mixture is stirred at ambient temperature for 12 hours. The reaction medium is heated at reflux for 4 hours. After evaporation of the solvents, the residue is diluted with dichloromethane and washed with a saturated solution of NaCl. The organic phase is separated, dried over magnesium sulphate, filtered and concentrated under vacuum. The expected product is crystallized in the form of a white solid. Melting point: 204° C.

### Stage 4

### 2,6-ditert-butyl-4-{2-[2-(methylamino)ethyl]-1,3-thiazol-4-yl}phenol hydrochloride

1.95 mmol of the intermediate obtained in Stage 3 is dissolved in ethyl acetate (20 ml). The solution is cooled down to 0° C. then HCl gas is bubbled through for 10 minutes. The mixture is left to return to ambient temperature while stirring is maintained. After filtration and drying under vacuum, the expected product is recovered in the form of white crystals which are washed with ether. Quantitative yield. Melting point: 206-208° C.

### Preparation 15

### 2,6-ditert-butyl-4-[2-(methoxymethyl)-1,3-thiazol-4-yl]phenol

### Stage 1

### [4-(3,5-ditert-butyl-4-hydroxyphenyl)-1,3-thiazol-2-yl]methyl pivalate

This compound is prepared according to a protocol identical to that described for Stage 3 of Preparation 14, using 2-(tert-butylcarbonyloxy)thioacetamide instead of the intermediate obtained in Stage 2 of Preparation 14 and with toluene replacing the benzene. The expected compound is obtained in the form of a white solid with a yield of 100%. Melting point: 114.6-116.0° C.

### Stage 2

### 2,6-ditert-butyl-4-[2-(hydroxymethyl)-1,3-thiol-4-yl]phenol

The intermediate obtained in Stage 1 (1.25 mmol) is dissolved in methanol (20 ml). The solution is cooled down using an ice bath then a 1N solution of NaOH is added dropwise. The mixture is left to return to ambient temperature while stirring. After evaporation to dryness and dilution of the residue with water, the solution is neutralized using citric acid and extraction is carried out with dichloromethane. The organic phase is washed with sodium chloride in a saturated aqueous solution before being dried over magnesium sulphate, filtered and concentrated under vacuum. A white solid is obtained with a yield of 88%. Melting point: 126.4-127.4° C.

### Stage 3

### 2,6-ditert-butyl-4-[2-(methoxymethyl)-1,3-thiazol-4-yl]phenol

The intermediate obtained in Stage 2 (1 equivalent) is methylated by reaction with 1.1 equivalent of iodomethyl in the presence of 2 equivalents of triethylamine, the reaction being carried out in tetrahydrofuran. A dark cream powder is obtained. Melting point: 115.8-117° C.

### Preparation 16

### 2,6-ditert-butyl-4-[2-(morpholin-4-ylmethyl)-1,3-thiazol-4-yl]phenol

### Stage 1

### 4-[2-(bromomethyl)-1,3-thiazol-4-yl]-2,6-ditert-butylphenol

1.5 g (4.70 mmol) of the intermediate obtained in Stage 2 of Preparation 15, (2,6-ditert-butyl-4-[2-(hydroxymethyl)-1,3-thiazol-4-yl]phenol, are dissolved in dichloromethane (30 ml). After adding CBr₄ (2.02 g; 6.10 mmol), the reaction medium is cooled down to 0 CC. PPh₃ (1.48 g; 5.63 mmol) is added by fractions then the mixture is allowed to return to ambient temperature. The reaction medium is then poured into ice-cold water before being extracted with dichloromethane. The organic phase is washed with salt water before being dried over magnesium sulphate, filtered and concentrated under vacuum. The expected product is obtained after chromatography on a silica column (eluent: 30% of ethyl acetate in heptane), in order to produce a brown oil with a yield of 92%. This product is sufficiently pure to be able to be used directly in the following stage.

MH+=382.20.

### Stage 2

### 2,6-ditert-butyl-4-[2-(morpholin-4-ylmethyl)-1,3-thiazol-4-yl]phenol

1.57 mmol of morpholine and 0.4 ml (2.62 mmol) of triethylamine are dissolved in dimethylformamide (15 ml). 0.400 g (1.05 mmol) of the intermediate obtained in Stage 1 dissolved in dimethylformamide (5 ml) is added then the mixture is stirred at ambient temperature for 18 hours. The reaction medium is then poured into ice-cold water and extraction is carried out with ethyl acetate. The organic phase is washed with salt water before being dried over magnesium sulphate, filtered and concentrated under vacuum. The expected product is obtained after chromatography on a silica column (eluent: 50% ethyl acetate in heptane), in order to produce an orange oil with a yield of 92%. Light cream crystals are obtained, Melting point: 136.7-137.2° C.

### Example 1

### butyl 2-[4-(4-aminophenyl)-1H-imidazol-2-yl]ethylcarbamate

### 1.1) butyl 2-[4-(4-nitrophenyl)-1H-imidazol-2-yl]ethylcarbamate

This compound is prepared according to a protocol identical to that described for Preparation 9,4-nitrophenacyl bromide replacing 4-phenyl-bromoacetophenone in Stage 2. The expected product is obtained in the form of brown powder with a yield of 1%.

MH+=333.20.

### 1.2) butyl 2-[4-(4-aminophenyl)-1H-imidazol-2-yl]ethylcarbamate

Intermediate 1.1 (0.28 g, 0.84 mmol) is dissolved in 20 ml of ethanol. 0.02 g of palladium on carbon (10%) is added and the mixture is placed under a hydrogen atmosphere (pressure of 2 bars). The catalyst is recovered by filtration then the solvent is evaporated off under reduced pressure. The expected product is purified by chromatography on a silica column (eluent=8% methanol and 0.5% ammonia in dichloromethane) in order to produce a brown powder with a yield of 24%. Melting point: 120° C.

### Example 2

### N,2-dimethyl-1-[4-(10H-phenothiazin-2-yl-1,3-thiazol-2-yl]propan-1-amine

### 2.1) N-[(benzyloxy)carbonyl]-N-methylvaline

10.0 g (0.0762 mol) of N-(Me)-(DL)-Valine-OH is dissolved in a dioxane/water mixture (90/10; 100 ml) and pH is adjusted to 11 using a 1N sodium hydroxide aqueous solution. Benzyloxysuccinimide (20.9 g; 0.0839 mol) in dioxane (40 ml) is added dropwise and the mixture is stirred overnight at room temperature. The reaction medium is then poured into ice-cooled water and acidified using a 10% aqueous citric acid solution before being extracted with ethyl acetate. The organic phase is washed with a saturated solution of sodium chloride. The organic phase is then dried over magnesium sulphate, filtered and concentrated under vacuum. The residue is purified on a silica column (eluent: 5% ethanol in dichloromethane) producing the title compound in the form of a pale yellow oil with a yield of 64%

MH+=266.10.

### 2.2) N-(Methyl)(CBZ)-(DL)-Valine-NH₂

Hydroxybenzotriazole (100 g; 0.653 mol) is suspended in methanol (500 ml), aqueous ammonium hydroxide 28% (60 ml) is added dropwise at ambient temperature and the suspension slowly goes into solution before precipitation, stirring being continued for approximately 5 hours. The methanol is evaporated off and the white solid triturated with isopropylether. The solid is filtered and washed with isopropyl ether in order to produce the HOBT.NH₃ complex in the form of a white powder with a 76% yield.

Intermediate 2.1 (12.9 g; 0.0486 moldy HOBT.NH₃ (as prepared previously; 9.1 g; 0.0584 mol) and benzotriazol-1-yloxytris(dimethylamino)phosphoniumhexafluorophosphate (BOP) (21.5 g; 0.0486 mol) are dissolved in DMF (120 ml) under an argon atmosphere, The mixture is cooled down to 0° C. and di-isopropylethylamine (18.7 ml) is added dropwise. The reaction medium is left to return to ambient temperature with stirring overnight. The reaction medium is then poured into ice-cooled water and extraction with ethyl acetate carried out. The organic phase is washed with a 10% aqueous sodium bicarbonate solution followed by a saturated solution of sodium chloride. The organic phase is then dried over magnesium sulphate, filtered and concentrated under vacuum. The solid residue is triturated with ether, the solid is filtered to produce a white hygroscopic solid with a yield of 83%, which is used directly in the next stage.

MH+=265.20

### 2.3) Benzyl 1-(aminocarbonothioyl)-2-methylpropyl(methyl)carbamate

This compound is prepared according to a protocol identical to that described for Stage 1.2 of Example 1, intermediate 2.2 replacing intermediate 1.1. The expected product is obtained in the form of a white solid with a yield of 36%. Melting point: 130° C.

### 2.4) Benzyl methyl {2-methyl-1-[4-(10H-phenothiazin-2-yl)-1,3-thiazol-2-yl]propyl}carbamate

This compound is prepared according to a protocol identical to that described for Stage 1.3 of Example 1, intermediate 2.3 replacing intermediate 1.2, 2-chloro-1-[10-chloroacetyl)-10H-phenothiazin-2-yl)ethanone replacing bromo-1-(3,5-ditert-butyl-4-hydroxyphenyl)ethanone and toluene replacing benzene. The expected product is obtained in the form of a yellow-orange foam with a yield of 49%.

MH+=502.10.

### 2.5) N,2-dimethyl-1-[4-(10H-phenothiazin-2-yl)-1,3-thiazol-2-yl]propan-1-amine

Intermediate 2.4 (1.2 g; 0.00238 mol) is dissolved in glacial acetic (12 ml). Concentrated HCl (4 ml) is added dropwise and the mixture is then heated at 100° C. for 2 hours before being evaporated to dryness. The residue is taken up in dichloromethane and washed with a 10% aqueous sodium bicarbonate solution then with saturated solutions of sodium chloride until the aqueous phase is neutral (pH paper). The organic phase is then dried over magnesium sulphate, filtered and concentrated under vacuum. The residue is purified on an reversed-phase silica column RP 18 (eluent: 40% aqueous (0.1N) TFA in acetonitrile). The combined fractions are evaporated to dryness, a 10% aqueous sodium bicarbonate solution is added to the residue and the mixture extracted with dichloromethane then with a saturated solution of sodium chloride. The organic phase is then dried over magnesium sulphate, filtered and concentrated under vacuum. The solid is triturated with isopentane to produce the title compound in the form of a yellow-orange solid with a yield of 14%. Melting point: 143.2-144.0° C.

### Example 3

### N,2-dimethyl-1-[4-10H-phenoxazin-2-yl)-1,3-thiazol-2-yl]propan-1-amine

The experimental protocol used is identical to that described for Example 2, 2-chloro-1-[10-(chloroacetyl)-10H-phenoxazine-2-yl)ethanone replacing 2-chloro-1-[10-(chloroacetyl)-10H-phenothiazin-2-yl)ethanone to finally produce the title compound in the form of a chestnut foam. MH+=352.2.

### Example 4

### N,3-dimethyl-1-[4-(10H-phenoxazin-2-yl)-1,3-thiazol-2-yl]butan-1-amine

The experimental protocol used is identical to that described for Example 2, thioamide benzyl I-(aminocarbonothioyl)-3-methylbutyl(methyl)carbamate (prepared in the same way as intermediate 2.3) replacing benzyl 1-(aminocarbonothioyl)-2-methylpropyl(methyl)carbamate and 2-chloro-1-[10-(chloroacetyl)-10H-phenoxazine-2-yl)ethanone replacing 2-chloro-1-[10-(chloroacetyl)-10H-phenotiazin-2-yl)ethanone in Stage 2.4 in order to finally produce the title compound in the form of a beige solid. Melting point: 143.1-147.0° C.

### Example 5

### N,3-dimethyl-1-[4-(10H-phenothiazin-2-yl)-1,3-thiazol-2-yl]butan-1-amine

The experimental protocol used is identical to that described for Example 2, thioamide benzyl-1-(aminocarbonothioyl)-3-methylbutyl(methyl)carbamate (prepared in the same way as the intermediate 2.3) replacing benzyl 1-(aminocarbonothioyl)-2-methylpropyl(methyl)carbamate in Stage 2.4, in order to finally produce the title compound in the form of yellow crystals. Melting point: 145.7-148.1° C.

### Example 6

### hydrochloride salt of 2,6-di-tert-butyl-4-{2-[3-methyl-1-(methylamino)butyl]-1,3-thiazol-4-yl}phenol

The experimental protocol used is identical to that described for Example 2, thioamide benzyl-1-(aminocarbonothioyl)-3-methylbutyl(methyl)carbamate prepared in the same way as the intermediate 2.3) replacing benzyl 1-(aminocarbonothioyl)-2-methylpropyl(methyl)carbamate and 2-bromo-1-(3,5-ditert-butyl-4-hydroxyphenyl)ethanone replacing 2-chloro-1-[10-(chloroacetyl)-10H-phenothiazin-2-yl)ethanone in Stage 2.4 during which removal of the CBZ protective group occurred in situ. The resulting free base compound is purified by normal phase chromatography on a silica-gel column (eluent: 30% ethyl acetate in heptane). After treatment of the free base using 1N HCl in ether, the title compound is obtained in the form of a creamy-white solid with an overall yield of 13%. Melting point: 148.1-149.0° C.

### Example 7

### hydrochloride salt of [4-(3,5-di-ter-butylphenyl)-1,3-thiazol-2-yl]methylamine

The experimental protocol used is identical to that described for Example 2, thioamide benzyl 2-amino-2-thioxoethylcarbamate (prepared in the same way as intermediate 2.3) replacing intermediate 2.3 and 2-bromo-1-(3,5-ditert-butyl-phenylethanone replacing 2-chloro-1-[10-(chloroacetyl)-10H-phenothiazin-2-yl)ethanone in Stage 2.4. The deprotection of the CBZ protective group is carried out in the same way as in Stage 2.5. The formed free base compound is purified by normal phase chromatography on a silica-gel column (eluent: 10% ethyl acetate in heptane) After treatment of the free base using 1N HCl in ether, the title compound is obtained in the form of a creamy-white solid. Melting point: 207.0-209.6° C.

### Example 8

### hydrochloride salt of 2,6-di-tert-butyl-4-{2-[(1S)-1-(methylamino)ethyl]-1,3-thiazol-4-yl}phenol

The experimental protocol used is identical to that described for Example 2, thioamide benzyl (1S)-2-amino-1-methyl-2-thioxoethyl(methyl)carbamate replacing intermediate 2.3 and 2-bromo-1-(3,5-ditert-butyl-4-hydroxyphenyl)ethanone replacing 2-chloro-1-[10-(chloroacetyl)-10H-phenothiazin-2-yl)ethanone. The deprotection of the (CBZ) protective group is carried out in the same way as in stage 2.5. The formed free base compound is purified by normal phase chromatography on a silica-gel column (eluent: 3% ethanol in dichloromethane). After treatment of the free base using 1N HCl in ether, the title compound is obtained in the form of a white crystalline solid. Melting point: 240.6-242.0° C.

### Example 9

### hydrochloride salt of 2,6-di-tert-butyl-4-{2-[(1R)-1-(methylamino)ethyl]-1,3-thiazol-4-yl}phenol

The experimental protocol used is identical to that described for Example 8, thioamide benzyl (1R)-2-amino-1-methyl-2-thioxoethyl(methyl)carbamate replacing thioamide benzyl (1S)-2-amino-1-methyl-2-thioxoethyl(methyl)carbamate, in order to finally produce, after formation of the salt, the title compound as a white crystalline solid. Melting point: 242.8-243.6° C.

### Example 10

### hydrochloride salt of N-{[4-(3,5-di-tert-butylphenyl)-1,3-thiazol-2-yl]methyl}-N-methylamine

The experimental protocol used is identical to that described for Preparation 1, 2-bromo-1-(3,5-ditert-butyl-phenyl)ethanone replacing 2-bromo-1-(3,5-ditert-butyl-4-hydroxyphenyl)ethanone in Stage 3. Elimination of the N-(Boc) protective group and formation of the salt are carried out in one stage using HCl gas according to a protocol similar to that described for Preparation 7, Stage 2 in order to produce the title compound in the form of a creamy-white solid. Melting point: 212.2-213.9° C.

### Example 11

### hydrochloride salt of N-methyl-N-{[4-(3,4,5-trimethoxyphenyl)-1,3-thiazol-2-yl]methyl}amine

### 11.1) 2-bromo-1-(3,4,5-trimethoxyphenyl)ethanone

The experimental protocol used is identical to that described for Preparation 12, Stage 2, commercially available 3,4,5-trimethoxy-acetophenone replacing the intermediate obtained in Stage 1 of Preparation 12. Intermediate 11.1 is obtained after chromatography on a silica column (eluent: 50% ethyl acetate in heptane) in the form of a yellow solid with a yield of 66%.

MH+=289.01

### 11.2) Hydrochloride salt of N-methyl-N-{[4-(3,4,5-trimethoxyphenyl)-1,3-thiazol-2-yl]methyl}amine

This compound is obtained using the same protocol as that described for Preparation 1, intermediate 11.1 replacing 2-bromo-1-(3,5-ditert-butyl-4-hydroxyphenyl)ethanone in Stage 4. Elimination of the Boc protective group and formation of the salt were carried out in one stage using HCl gas according to a protocol similar to that described for Preparation 7, Stage 2 in order to produce the title compound in the form of a yellow crystalline solid. Melting point: 199.4-200.6° C.,

### Example 12

### hydrochloride salt of ethyl N-{[4-(3,5-di-tert-butyl-4-hydroxyphenyl %1,3-thiazol-2-yl]methyl}glycinate

The experimental protocol used is identical to that described for Preparation 2; ethyl bromoacetate replacing chloropropargyl and the compound of Preparation 4 replacing the compound of Preparation 1. After treatment of the free base using 1N HCl in ether, the title compound is obtained in the form of a white crystalline solid with an overall yield of 69%. Melting point: 164.0-167.0° C.

### Example 13

### hydrochloride salt of N-{[4-(3,5-di-tert-butyl-4-hydroxyphenyl)-1,3-thiazol-2-yl]methyl}glycine

### 13.1) Ethyl N-(tert-butoxycarbonyl)-N-{[4-(3,5-di-tert-butyl-4-hydroxyphenyl)-1,3-thiazol-2-yl]methyl}glycinate

The experimental protocol used is identical to that described for Preparation 14, Stage 1, the compound of Example 12 replacing N-methyl-β-alaninenitrile, triethylamine being used instead of diisopropylethylamine and a catalytic amount of dimethylaminopyridine (DMAP) being added to carry out the reaction. The title compound is obtained in the form of a green oil which is used directly in the next stage.

MH+=505.30.

### 13.2) N-(tert-butoxycarbonyl)-N-{[4-(3,5-di-tert-butyl-4-hydroxyphenyl)-1,3-thiazol-2-yl]methyl}glycine

1.0 g (1.98 mmol) of intermediate 13.1 is dissolved in THF (20 ml). A solution of lithium hydroxide (1N in water) is added dropwise and the reaction medium is left under stirring at ambient temperature for 6 hours. The reaction medium is then poured into water followed by extraction with diethyl ether. The aqueous phase is acidified with aqueous HCl (1N) and extracted with diethyl ether. The organic phase is washed with a saturated solution of sodium chloride, then dried over magnesium sulphate, filtered and concentrated under vacuum before being used directly in the next stage.

MH+=477.20.

### 13.3) Hydrochloride salt of N-{[4-(3,5-di-tert-butyl-4-hydroxyphenyl)-1,3-thiazol-2-yl]methyl}glycine

The experimental protocol used is identical to that described for Preparation 7, Stage 2, intermediate 13.2 replacing the intermediate obtained in Stage 1 of Preparation 7. The title compound is obtained in the form of a white crystalline solid with a yield of 27%.

MH+=377.2.

### Example 14

### hydrochloride salt of 2,6-di-tert-butyl-4-{2-[(4-methoxypiperidin-1-yl)methyl]-1,3-thiazol-4-yl}phenol

The experimental protocol used is identical to that described for Preparation 16, Stage 2,4-methoxy-piperidine replacing morpholine. The title compound is obtained in the form of a white crystalline solid. Melting point: 198.0-201.0° C.

### Example 15

### hydrochloride salt of N-methyl-N-{(1S)-2-methyl-1-[4-10H-phenothiazin-2-yl)-1,3-thiazol-2-yl]propyl}amine

This compound is prepared in an analogous way to the compound of Example 2, however using optically pure starting material, namely N-(Me)-(L)-Valine-OH instead of N-(Me)-(DL)-Valine-OH). Melting point: 270.0-270.8° C.

### Example 16

### hydrochloride salt of N,2-dimethyl-1-[4-(10-methyl-10H-phenothiazin-2-yl)-1,3-thiazol-2-yl]propan-1-amine

### 16.1) Benzylmethyl{2-methyl-1-[4-(10-methyl-10H-phenothiazin-2-yl)-1,3-thiazol-2-yl]propyl}carbamate

Intermediate 2.4 was methylated according to the following procedure: 0.200 g (0.410 mmol) of intermediate 2.4 is dissolved in dioxane (10 ml). Sodium hydride (0.024 g; 0.598 mmol) is added in small portions and the mixture is left under stirring for 30 minutes. Iodomethane (0.04 ml) is added dropwise and the reaction medium is heated at 45 C for 18 hours. Ethanol (10 ml) is added dropwise and the reaction medium is then poured into water before being extracted with ethyl acetate. The organic phase is washed with a saturated solution of sodium chloride, then dried over magnesium sulphate, filtered and concentrated under vacuum. Intermediate 16.1 is obtained after chromatography on a silica column (eluent: 15% ethyl acetate in heptane) as a yellow gummy solid with a yield of 42%.

MH+=516.10.

### 16.2) Hydrochloride salt of N,2-dimethyl-1-[4-(10-methyl-10H-phenothiazin-2-yl)-1,3-thiazol-2-yl]propan-1-amine

Intermediate 16.1 is dissolved in a glacial acetic acid/water/methanol (30 ml) mixture. A catalytic amount of Pd/C is added and the reaction medium is hydrogenated at ambient temperature under a pressure of 5 bars for 12 hours. The exhausted catalyst is filtered off and the filtrate was evaporated to dryness and azeotropic distillation with toluene is carried out several times. The hydrochloride salt, a grey solid, is obtained using a 1N HCl solution in ether with an overall yield of 45%.

MH+=382.10.

### Example 17

### hydrochloride salt of N-methyl-N-{(1S)-2-methyl-1-[4-(10H-phenoxazin-2-yl)-1,3-thiazol-2-yl]propyl}amine

This compound is prepared in an analogous way to the compound of Example 3, however using optically pure starting material, namely N-(Me)-(CBZ)-(L)-Valine-OH instead of N-(Me)-DL)-Valine-OH. A grey powder is obtained.

MH+=352.2.

### Example 18

### hydrochloride salt of 4-{2-[(1R)-1-aminoethyl]-1,3-thiazol-4-yl}-2,6-di-tert-butylphenol

The experimental protocol used is identical to that described for the compound of Example 2, thioamide benzyl (1R)-2-amino-1-methyl-2-thioxoethylcarbamate (prepared in a similar way to the intermediate 2.3) replacing thioamide benzyl 1-(aminocarbonothioyl)-2-methylpropyl(methyl)carbamate and 2-bromo-1-(3,5-ditert-butyl-4-hydroxyphenyl)ethanone replacing 2-chloro-1-[10-(chloroacetyl)-10H-phenothiazin-2-yl)ethanone in the fourth stage. The title hydrochloride salt is then obtained as a white solid using 1N HCl in ether. Melting point: 211.8-215.2° C.

### Example 19

### hydrochloride salt of 4-{2-[(1S)-1-aminoethyl]-1,3-thiazol-4-yl}-2,6-di-tert-butylphenol

The experimental protocol used is identical to that described for Example 2, thioamide benzyl (1S)-2-amino-1-methyl-2-thioxoethylcarbamate (prepared in a similar way to the intermediate 2.3) replacing thioamide benzyl 1-(aminocarbonothioyl)-2-methylpropyl(methyl)carbamate and 2-bromo-1-(3,5-ditert-butyl-4-hydroxyphenyl)ethanone replacing 2-chloro-1-[10-(chloroacetyl)-10H-phenothiazin-2-yl)ethanone in the fourth stage. The title hydrochloride salt is then obtained as a white solid using 1N HCl in ether. Melting point: 191.0-195.0° C.

### Example 20

### hydrochloride salt of 4-[2-(1-aminocyclopropyl)-1,3-thiazol-4-yl]-2,6-di-tert-butylphenol

### 20.1) tert-butyl [1-(aminocarbonyl)cyclopropyl]carbamate

The experimental protocol used is identical to that described for Stage 2 of Example 2, 1-[(tert-butoxycarbonyl)amino]cyclopropanecarboxylic acid replacing intermediate 2.1.

The title compound is obtained in the form of a colourless oil used directly in the next stage.

### 20.2) Tert-butyl [1-(aminocarbonothioyl)cyclopropyl]carbamate

This compound is prepared according to a protocol identical to that described for Stage 2 of Preparation 1, intermediate 20.1 replacing the intermediate obtained in Stage 1 of Preparation 1. The expected product is obtained in the form of a yellow oil used directly in the next stage.

### 20.3) Hydrochloride salt of 4-[2-(1-aminocyclopropyl)-1,3-thiazol-4-yl]-2,6-di-tert-butylphenol

This compound is prepared according to a protocol identical to that described for Stage 3 of Preparation 1, intermediate 20.2 replacing the intermediate obtained in Stage 2 of Preparation 1 and toluene replacing benzene. Deprotection of the N-(Boc) protective to group occurs in-situ The hydrochloride salt is then obtained in the form of a white-creamy solid using 1N HCl in ether Melting point: 200.6-202.2° C.

### Example 21

### hydrochloride salt of 4-{2-[(methylamino)methyl]-1,3-thiazol-4-yl}benzene-1,2-diol

The experimental protocol used is identical to that described for Preparation 1, 2-chloro-3′,4′-dihydroxyacetophenone replacing 2-bromo-1-(3,5-ditert-butyl-4-hydroxyphenyl)ethanone. The title compound is obtained in the form of a white-creamy solid.

MH+=237.0.

### Example 22

### hydrochloride salt of N-methyl-N-{(1R)-2-methyl-1-[4-(10H-phenothiazin-2-yl)-1,3-thiazol-2-yl]propyl}amine

This compound is prepared in an analogous way to Example 2, however using optically pure starting material, i.e. N-(Me)-(CBZ)-(D)-Valine-OH instead of N-(Me)-(DL)-Valine-OH. The title compound is obtained in the form of a light green powder. Melting point: 265.6-268.9° C.

### Example 23

### hydrochloride salt of (1R)-2-methyl-1-[4-(10H-phenothiazin-2-yl)-1,3-thiazol-2-yl]propan-1-amine

### 23.1) tert-butyl(1R)-[4-(10-acetyl-10H-phenothiazin-2-yl)-1,3-thiazol-2-yl]-2-methylpropylcarbamate

The experimental protocol used is identical to that described for Preparation 1, 1-(10-acetyl-10H-phenothiazin-2-yl)-2-bromoethanone (Arzneimittel Forschung (1962), 12, 48-52) replacing 2-bromo-1-(3,5-ditert-butyl-4-hydroxyphenyl)ethanone and thioamide tert-butyl (1R)-1-(aminocarbonothioyl)-2-methylpropylcarbamate (prepared in a similar way to the intermediate obtained in Stage 2 of Preparation 1) replacing the intermediate obtained in Stage 2 of Preparation 1. Elimination of the N-(Boc) protective group and formation of the salt are carried out in one stage using HCl gas according to a procedure similar to that described for Stage 2 of Preparation 7. The title compound is obtained in the form of a grey solid.

MH+=396.1.

### 23.2) Hydrochloride salt of (1R)-2-methyl-1-[4-(10H-phenothiazin-2-yl)-1,3-thiazol-2-yl]propan-1-amine

Intermediate 23.1 is dissolved in 2N HCl and refluxed for 18 hours. The solution is extracted with ethyl acetate and the aqueous phase is made basic using an aqueous solution (10%) of sodium bicarbonate and extracted with ethyl acetate. The organic phase is washed with a 10% aqueous sodium bicarbonate solution then with a saturated solution of sodium chloride. The organic phase is dried over magnesium sulphate, filtered and concentrated under vacuum. The residue is purified on a silica column (eluent: 5% ethanol in dichloromethane), producing the free base as a pale brown oil. The hydrochloride salt is obtained in the form of a green powder using 1N HCl in ether.

MH+=354.2.

### Example 24

### hydrochloride salt of N-methyl-N-{(1R)-2-methyl-1-[4-10H-phenoxazin-2-yl)-1,3-thiazol-2-yl]propyl}amine

This compound is prepared in an analogous way to Stages 2.2 to 2.5 of Example 2, however using optically pure starting material, namely N-(Me)-(CBZ)-(D)-Valine-OH instead of intermediate 2.1. The title hydrochloride salt is obtained as in the form of a yellow powder,

MH+=352.2.

### Example 25

### hydrochloride salt of N²-{[4-(3,5-di-tert-butyl-4-hydroxyphenyl)-1,3-thiazol-2-yl]methyl}glycinamide

The experimental protocol used is identical to that described for Preparation 2, 2-bromoacetamide replacing chloropropargyl and the compound of Preparation 4 replacing the compound of Preparation 1. The hydrochloride salt is then obtained in the form of a white powder using 1N HCl in ether.

MH+=376.2.

### Example 26

### hydrochloride salt of ethyl N-{[4-(3,5-di-tert-butyl-4-hydroxyphenyl)-1,3-thiazol-2-yl]methyl}-N-(2-ethoxy-2-oxoethyl)glycinate

The experimental protocol used is identical to that described for Preparation 2, with however an excess of ethyl bromoacetate replacing chloropropargyl and the compound of Preparation 4 replacing the compound of Preparation 1. The hydrochloride salt is then obtained in the form of a white foam using 1N HCl in ether.

MH+=491.2.

### Example 27

### hydrochloride salt of 4-(3,5-di-ten-butyl-4-methoxyphenyl)-2-(methoxymethyl)-1,3-thiazole

The experimental protocol used is identical to that described for Stage 16.1 of Example 16, the intermediate obtained in Stage 1 of Preparation 13 replacing intermediate 2.4 and THF replacing dioxane. The title compound is obtained in the form of a white solid with a yield of 38%. Melting point: 94.0-94.8° C.

### Example 28

### hydrochloride salt of 4-[2-(1-aminocyclopentyl)-1,3-thiazol-4-yl]2,6-di-tert-butylphenol

### 28.1) ter-butyl [1-(aminocarbonyl)cyclopentyl]carbamate

The experimental protocol used is identical to that described for Stage 2 of Example 2, 1-[(tert-butoxycarbonyl)amino]cyclopentanecarboxylic acid replacing intermediate 2.1. The title compound is obtained as a white flaky solid which is used directly in the next stage.

### 28.2) tert-butyl [1-(aminocarbonothioyl)cyclopentyl]carbamate

This compound is prepared according to a protocol identical to that described for Stage 2 of Preparation 1, intermediate 28.1 replacing the intermediate obtained in Stage 1 of Preparation 1. The expected product is obtained in the form of a white flaky solid which is used directly in the next stage.

MH+=245.2.

### 28.3) tert-butyl {1-[4-(3,5-di-tert-butyl-4-hydroxyphenyl)-1,3-thiazol-2-yl]cyclopentyl}carbamate

This compound is prepared according to a protocol identical to that described for Stage 3 of Preparation 1, intermediate 28.2 replacing the intermediate obtained in Stage 2 of Preparation 1 and toluene replacing benzene. The expected product is obtained in the form of a colourless oil.

MH+=473.4.

### 28.4) Hydrochloride salt of 4-[2-(1-aminocyclopentyl)-1,3-thiazol-4-yl]-2,6-di-tert-butylphenol

Elimination of the N-(Boc) protective group from intermediate 28.3 and formation of the salt are carried out in one stage using HCl gas according to a protocol similar to that described for Stage 2 of Preparation 7. The title compound is obtained in the form of a white crystalline solid. Melting point: 288.8-290.7° C.

Pharmacological Study of the Products of the Invention

Study of the Effects on the Bond of a Specific Ligand of MAO-B, [³H]Ro 19-6327

The inhibitory activity of the products of the invention is determined by measurement of their effects on the bond of a specific ligand of MAO-B, [³H]Ro 19-6327.

a) Mitochondrial Preparation of the Cortex of Rats

The mitochondrial preparation of the cortex of rats is carried out according to the method described in Cesura A M, Galva M D, Imhof R and Da Prada M, J. Neurochem. 48 (1987), 170-176. The rats are decapitated and their cortex is removed homogenized in 9 volumes of a 0.32 M sucrose buffer, buffered to pH 7.4 with 5 mM of HEPES, then centrifuged at 800 g for 20 minutes. The supernatants are recovered and the pellets are washed twice with the 0.32 M sucrose buffer as previously. The collected supernatants are centrifuged at 10000 g for 20 minutes. The pellets obtained are suspended in a Tris buffer (50 mM Tris, 130 mM NaCl, 5 mM KCl, 0.5 mM EGTA, 1 mM MgCl₂, pH 7.4) and centrifuged at 10000 g for 20 minutes. This stage is repeated twice, and the final pellet, corresponding to the mitochondrial fraction, is stored at −80° C. in the Tris buffer. The protein content of the preparation is determined by the Lowry methods

b) Bond of [³H]Ro 19-6327

100 μl of the mitochondrial preparation (2 mg protein/ml) are incubated for 1 hour at 37° C. in an Eppendorf tube, in the presence of 100 μl of [³H]Ro 19-6327 (33 nM, final concentration) and 100 μl of Tris buffer containing or not containing the inhibitors. The reaction is stopped by the addition of 1 ml of cold Tris buffer into each tube, then the samples are centrifuged for 2 minutes at 12000 g. The supernatants are removed by suction and the pellets washed with 1 ml of Tris buffer. The pellets are then solubilized in 200 μl of sodium dodecyl sulphate (20% weight/volume) for 2 hours at 70° C. The radioactivity is determined by counting the samples using liquid scintillation.

c) Results

The compound of Example 9 described above shows an IC₅₀ lower than 10 μM. Moreover, the compounds of Examples 8, 18 and 19 described above show an IC₅₀ lower than 20 μM.

Study of the Effects on Lipidic Peroxidation of the Cerebral Cortex of the Rat

The inhibitory activity of the products of the invention is determined by measuring their effects on the degree of lipidic peroxidation, determined by the concentration of malondialdehyde (MDA). The MDA produced by peroxidation of unsaturated fatty acids is a good indication of lipidic peroxidation (H Esterbauer and K H Cheeseman, Meth. Enzymol. (1990) 186: 407-421). Male Sprague Dawley rats weighing 200 to 250 g (Charles River) were sacrificed by decapitation. The cerebral cortex is removed, then homogenized using a Thomas potter in a 20 mM Tris-HCl buffer, pH=7.4. The homogenate is centrifuged twice at 50000 g for 10 minutes at 4° C. The pellet is stored at −80° C. On the day of the experiment, the pellet is resuspended at a concentration of 1 g/15 ml and centrifuged at 515 g for 10 minutes at 4° C. The supernatant is used immediately to determine the lipidic peroxidation. The homogenate of rat's cerebral cortex (500 μl) is incubated at 37° C. for 15 minutes in the presence of the compounds to be tested or of the solvent (10 μl). The lipidic peroxidation reaction is initiated by adding 50 μl of FeCl₂ at 1 mM, EDTA at 1 mM and ascorbic acid at 4 mM. After incubation for 30 minutes at 37° C., the reaction is stopped by adding 50 μl of a solution of hydroxylated di-tert-butyl toluene (BHT, 0.2%). The MDA is quantified using a colorimetric test, by reacting a chromogenic reagent (R), N-methyl-2-phenylindol (650 μl) with 200 μl of the homogenate for 1 hour at 45° C. The condensation of an MDA molecule with two molecules of reagent R produces a stable chromophore the maximum absorbence wavelength of which is equal to 586 nm (Caldwell et al., European J. Pharmacol. (1995), 285, 203-206). The compounds of Examples 2 to 6, 8, 9 and 12 to 26 described above show an IC₅₀ lower than 10 μM.

Bond Test on the Cerebral Sodium Channels of the Cortex of the Rat

The test consists in measuring the interaction of the compounds vis-à-vis the bond of tritiated batrachotoxin on the voltage-dependent sodium channels according to the protocol described by Brown (J. Neurosci. (1986), 6, 2064-2070).

### Preparation of Homogenates of Cerebral Cortices of the Rat

The cerebral cortices of Sprague-Dawley rats weighing 230-250 g (Charles River, France) are removed, weighed and homogenized using a Potter homogenizer provided with a teflon piston (10 strokes) in 10 volumes of isolation buffer the composition of which is as follows (sucrose 0.32 M; K₂HPO₄ 5 mM; pH 7.4). The homogenate is subjected to a first centrifugation at 1000 g for 10 minutes. The supernatant is removed and centrifuged at 20000 g for 15 minutes. The pellet is taken up in the isolation buffer and centrifuged at 20000 g for 15 minutes. The pellet obtained is resuspended in incubation buffer (HEPES 50 mM; KCl 5.4 mM; MgSO₄ 0.8 mM; glucose 5.5 mM; choline chloride 130 mM pH 7.4) then aliquoted and stored at −80° C. until the day of assay. The final protein concentration is comprised between 4 and 8 mg/ml. The assay of proteins is carried out using a kit marketed by BioRad (France).

Measurement of the Bond of Tritiated Batrachotoxin

The bond reaction is carried out by incubating for 1 hour 30 minutes at 25° C. 100 μl of homogenate of rat cortex containing 75 μg of proteins with 100 μl of [³H]batrachotoxin-A 20-alpha benzoate (37.5 Ci/mmol, NEN) at 5 nM (final concentration), 200 μl of tetrodotoxin at 1 mM (final concentration) and scorpion venom at 40 μg/ml (final concentration) and 100 μl of incubation buffer alone or in the presence of the products to be tested at different concentrations. The non-specific bond is determined in the presence of 300 μM of veratridine and the value of this non-specific bond is subtracted from all the other values. The samples are then filtered using a Brandel (Gaithersburg, Md., USA) using Unifilter GF/C plates pre-incubated with 0.1% of polyethylene imine (20 μl/well) and rinsed twice with 2 ml of filtration buffer (HEPES 5 mM; CaCl₂ 1.8 mM; MgSO₄ 0.8 mM; choline chloride 130 mM; BSA 0.01%; pH 7.4). After having added 20 μl of Microscint 0®, the radioactivity is counted using a liquid scintillation counter (Topcount, Packard). The measurement is carried out in duplicate. The results are expressed as a % of the specific bond of tritiated batrachotoxin relative to the control.

Results

The compounds of Examples 1, 8 to 10 and 25 described above all show an IC₅₀ lower than or equal to 1 μM.

1. A compound:
butyl 2-[4-(4-aminophenyl)-1H-imidazol-2-yl]ethylcarbamate;
or salts or combinations thereof.

2. (canceled)

3. (canceled)

4. A medicament, comprising a compound:
butyl 2-[4-(4-aminophenyl)-1H-imidazol-2-yl]ethylcarbamate;
or pharmaceutically acceptable salts or combinations thereof.

5. A pharmaceutical composition comprising, as an active ingredient, at least one of the compounds or pharmaceutically acceptable salts of claim 4.

6. (canceled)

7. (canceled)

8. (canceled)

9. (canceled)

10. (canceled)
